(19) United States
(12) Patent Application Publication
(10) Pub. No.: US 2025/0238009 A1
(43) Pub. Date: Jul. 24, 2025

(54) STERILIZABLE DEVICE FOR LOCATING STERILE GOODS

(71) Applicant: Aesculap AG, Tuttlingen (DE)

(72) Inventors: Matthias Schweizer, Tuttlingen (DE); Andreas Elisch, Dunningen (DE); Roland-Alois Hoegerle, Tuttlingen (DE); Frederick Lenzenhuber, Tuttlingen (DE)

(21) Appl. No.: 19/033,691

(22) Filed: Jan. 22, 2025

(30) Foreign Application Priority Data
Jan. 24, 2024 (DE) 10 2024 101 996.4

(51) Int. Cl. G05B 15/02; A61L 2/07; A61L 2/26

(52) U.S. Cl. CPC G05B 15/02; A61L 2/26; A61L 2/07

(57) ABSTRACT

A sterilizable device for locating sterile goods includes an energy source and electronic system for transmitting a locating signal. The energy source is configured to supply the electronic system with energy. The device includes a controller configured to detect a washing or sterilizing process and deactivate at least part of the electronic system at least temporarily in the washing or sterilizing process.

### CROSS-REFERENCE TO RELATED APPLICATION

This application claims priority under 35 U.S.C. § 119 to German Application No. 10 2024 101 996.4, filed on Jan. 24, 2024, the content of which is incorporated by reference herein in its entirety.

### FIELD

The invention is based on a sterilizable device for locating sterile goods.

### SUMMARY

A sterilizable device for locating sterile goods, in particular a power source for the device, should have the longest possible service life and be protected against the high temperatures that occur during a washing or sterilization process, as well as against water or superheated saturated water vapor that can lead to moisture on the device during the process, as well as against the pressure changes that occur and a pressure range from 30 mbar absolute to ˜3.1 bar absolute during the sterilization process.

In the present disclosure, a sterilizable device for locating sterile goods, comprising a power source and electronic system for transmitting a signal for locating, wherein the power source is configured to supply the electronic system with power, wherein the device comprises a controller that is configured to detect a process for washing or sterilizing and to at least temporarily deactivate at least part of the electronic system in the process.

The controller is preferably designed to detect a temperature and to deactivate at least part of the electronic system when a predetermined temperature threshold or a temperature threshold that can be predefined in particular via an interface of the device for setting the temperature is reached or exceeded.

The controller is preferably designed to activate at least part of the electronic system when a predetermined temperature threshold or a temperature threshold that can be predefined in particular via an interface of the device for setting the temperature is reached or undershot.

Preferably, the active part of the electronic system is designed to measure the temperature, wherein the controller is designed to activate the deactivated part of the electronic system at regular time intervals, to check whether the temperature falls below the temperature threshold value, and to deactivate at least the part of the electronic system if the temperature reaches or exceeds the temperature threshold value or otherwise to leave the deactivated part of the electronic system activated.

The controller is preferably designed to leave at least the part of the electronic system for a predetermined period of time, in particular in a firmware of the controller, or a time period that can be set in particular via an interface, in particular a data communication interface, or a mechanical element, preferably a switch or control dial, of the device for setting the time period after the detection of the process for sterilization, and to activate the deactivated part of the electronic system after the expiry of the time period in which at least the part of the electronic system is to be left deactivated, in particular at regular time intervals.

In one embodiment of the device, the device comprises an interface for signal communication, which is designed to receive a signal for activating at least part of the electronic system and/or which is designed to receive a signal for deactivating at least part of the electronic system, wherein the controller is configured to deactivate at least the part of the electronic system upon receipt of the deactivating signal, and/or wherein the controller is configured to activate at least the part of the electronic system upon receipt of the activating signal.

It may be provided that the signal for activation includes location information, wherein the controller is configured to activate the part of the electronic system that is activated depending on the location information and/or to deactivate the part of the electronic system that is deactivated depending on the location information.

The controller is preferably designed to activate at least part of the electronic system after a predetermined time or, in particular, after a time that can be specified via an interface of the device for setting the time after the event has been detected.

Preferably, the controller is designed to deactivate at least part of the electronic system by mechanically or electrically interrupting an electrical connection between the power source and at least part of the electronic system.

The controller is preferably designed to deactivate at least part of the electronic system by switching off a component of that part of the electronic system.

Preferably, the controller is designed to activate at least part of the electronic system by establishing an electrical connection between the energy source and at least part of the electronic system.

In one embodiment, the controller comprises a shape memory element, in particular a bimetal, which can assume at least a first shape and a second shape depending on the temperature, wherein the shape memory element is designed to deactivating at least part of the electronics by interrupting the electrical connection between the energy source and at least part of the electronic system, and activating at least part of the electronic system by establishing the electrical connection between the energy source and at least part of the electronic system in a second form of the shape memory element.

In one embodiment, the interface includes a coil, in particular a coil for near-field communication, preferably an NFC tag or RFID tag, wherein the controller is configured to detect a voltage induced in the coil and to activate at least the part of the electronic system when the induced voltage is detected.

In one embodiment, the controller includes a switching element that is designed to activate at least part of the electronic system, wherein the switching element is arranged on the device in such a way that the switching element is in communication or contact with a sterile supply container for the sterile supplies during the process. In this case, the switching element is designed to activate at least the part of the electronic system when contact is made or communication with the sterile supply container is established and to deactivate when contact is lost or communication with the sterile supply container is interrupted.

It may be provided that the switching element, in a first position of the switching element, allows a current provided by the energy source to flow through the switching element and, in a second position of the switching element, prevents a current from flowing through the switching element, wherein the controller is configured to distinguish the first position of the switching element from the second position of the switching element and to activate at least the part of the electronic system when the first position or a change from the second position to the first position is detected.

It may be provided that the switching element, in a first position of the switching element, allows a current provided by the energy source to flow through the switching element at least to part of the electronic system, and, in a second position of the switching element, prevents a current from flowing through the switching element at least part of the electronic system, wherein the switching element is designed to be in the first position when there is communication or contact with the sterile supply container and to move to the second position when there is a loss of communication or contact with the sterile supply container.

It may be envisaged that the electronic system comprises the part of the electronic system and another part, wherein the part of the electronic system is designed to be open and/or flushable in the process, and the other part of the electronic system is encapsulated, or wherein the electronic system is open and/or flushable in the process and the energy source is encapsulated.

### BRIEF DESCRIPTION OF THE DRAWING FIGURES

Further advantageous embodiments can be found in the following description and drawing figures, of which:

FIG. 1 is a schematic representation of a device for locating sterile goods;

FIG. 2 is a schematic representation of a process for sterilizing sterile goods;

FIG. 3 is a pressure curve and a temperature curve during a process for sterilizing sterile goods; and

FIG. 4 is a flow chart with steps of a method for operating the device.

### DETAILED DESCRIPTION

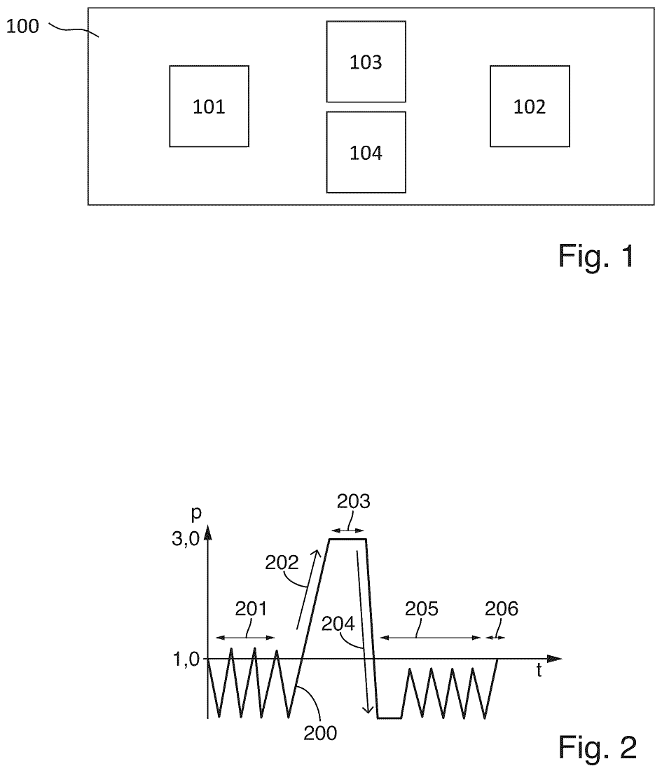

FIG. 1 schematically shows a device (100) for locating sterile items.

The device 100 is designed to be sterilized in and/or on a sterile goods container or in and/or on a disposable fleece packaging for sterile goods. The device 100 can be attached to the sterile supply container or inserted into the sterile supply container or disposable fleece packaging.

The device 100 comprises a power source 101, an electronic system 102 for sending a signal for localization by other devices or a signal for localization that includes the location of the sterile supply container or the disposable fleece packaging, and a controller 103. The device 100 may be provided with an interface 104.

The signal for localization can be a beacon signal that enables the other devices to locate it, e.g. by means of triangulation. The device 100 can be designed to receive location-determining signals from other devices and to determine the location depending on the location-determining signals, e.g. by triangulation.

The energy source 101 is, for example, a battery or an accumulator.

The electronic system 102 comprises at least one part that can be deactivated.

The electronic system 102 can comprise one or more parts that can be deactivated. The electronic system 102 may include a non-deactivatable part.

For example, to protect the electronic system 102 in the process, a part of the electronic system 102 that cannot be deactivated is encapsulated. The deactivatable part of the electronic system 102 or the deactivatable parts of the electronic system 102 can be open or designed with gaps that can be flushed through during the process. This offers great advantages over a complex complete encapsulation of the electronic system 102.

The electronic system 102 includes, for example, components such as a printed circuit board or a sensor, e.g. a temperature sensor, or a microcontroller or capacitors or coils or resistors or digital signal processors for receiving or generating signals.

The electronic system 102 includes, for example, components such as a printed circuit board or a sensor, e.g. a temperature sensor, or a microcontroller or capacitors or coils or resistors or digital signal processors for receiving or generating signals.

The energy source 101 is encapsulated in an embodiment. This provides an extra level of protection for the energy source 101 against short circuits and/or excessive temperatures. This also allows the use of a power source 101 that is not specifically designed for the high temperatures and humidity in the sterilization process. The energy source 101 is open in an embodiment. This requires the use of an energy source 101 that is specially designed for the high temperatures and humidity in the sterilization process and eliminates the need for encapsulation of the energy source 101.

This means that the device includes, for example, an encapsulated, highly protected area and a less protected, more open area or one with more simply designed housing. The less protected area does not necessarily have to be open. It can also be a simpler designed housing as in the highly encapsulated area.

Encapsulation of the protected areas is achieved, for example, by enclosing them in a stainless steel housing or a stainless steel and plastic insert in the area of the antenna. Encapsulation of the protected areas is achieved, for example, by enclosing them in a plastic housing that is resistant to alkaline cleaning chemicals, such as PPSU or PEEK. Encapsulation of the protected areas is achieved, for example, by embedding them in epoxy resin. The protected areas are protected from high process temperatures, for example, by insulation, for example with PU foam.

The electronic system 102 is designed to generate a signal to locate the device 100. The electronic system 102 is trained to send the signal.

The electronic system 102 includes, for example, an electrical circuit designed to generate the signal and an antenna to transmit the signal.

The energy source 101 is designed to supply the electronic system 102 with energy.

The controller 103 is designed to recognize a washing or sterilization process and to at least temporarily deactivate at least some of the electronic system 102 during the process.

According to one embodiment, the controller 103 is designed to detect a temperature and to deactivate at least the part of the electronic system 102 when a threshold value is reached or exceeded.

For example, the electronic system 102 includes a temperature sensor adapted to sense the temperature in the environment of the device 100.

The threshold can be predetermined or specifiable and is preferably a temperature threshold.

Alternatively or additionally, the electronic system 102 comprises a pressure sensor that is designed to detect the pressure in the environment of the device 100 and the predetermined or predeterminable threshold value is a pressure threshold value.

The controller 103 includes, for example, a memory in which the temperature threshold value is stored. The controller 103 includes, for example, a microprocessor that includes or accesses the memory and is configured to detect that the temperature reaches or exceeds the temperature threshold and to disable the electronic system 102 when the temperature reaches or exceeds the temperature threshold.

The specified temperature threshold value is stored in the memory, for example in firmware. It may be possible to specify the temperature threshold via the interface 104. The interface 104 includes, for example, a data communication interface or a mechanical element, preferably a switch or dial, for setting the temperature threshold.

It may be envisaged that the part of the electronic system 102 that is deactivated is designed to detect the temperature. This is particularly advantageous if the deactivatable part of the electronic system is activated or reactivated after a predetermined or predeterminable time after its deactivation, without taking into account a further temperature threshold.

In one embodiment, the controller 103 is designed to activate at least part of the electronic system 102 only after a certain time has elapsed after the sterilization process has been detected or after the temperature threshold has been reached.

The time can be specified or specifiable.

Time, for example, is stored in memory. The microprocessor is designed, for example, to recognize the passage of time and to activate the electronic system 102 for testing only when the passage of time is recognized.

The specified time is stored in the memory, e.g. in the firmware.

It may be possible to specify the time via the interface 104. For example, the time can be set via the data communication interface or a mechanical element, preferably a switch or control dial, can be used to set the time.

The controller 103 is designed, for example, to activate the deactivated part of the electronic system 102 at regular time intervals for a temperature check. The test checks, for example, whether the temperature reaches or exceeds or falls below the temperature threshold.

The controller 103 is designed, for example, depending on the result of the test, to at least deactivate the electronic system 102 again if the temperature reaches or exceeds the temperature threshold.

The controller 103 is designed, for example, depending on the result of the test, to leave the previously deactivated part of the electronic system 102 activated if the temperature is below the temperature threshold.

The microprocessor is designed, for example, to carry out the test and to disable or leave enabled at least part of the electronic system 102 depending on the result of the test.

It may be provided that the controller 103 is designed to deactivate at least the part of the electronic system 102 after recognizing the sterilization process and to activate the deactivated part of the electronic system 102 for testing only after a period of time has elapsed since the sterilization process was recognized.

The time span can be predefined or definable.

The time span, for example, is stored in the memory. The microprocessor is designed, for example, to recognize the expiration of the time period and to activate the electronic system 102 for testing for the first time when the expiration of the time period is recognized.

The specified time span is stored in the memory, e.g. in the firmware.

It may be possible to specify the temperature threshold via the interface 104. The interface 104 includes, for example, a data communication interface or a mechanical element, preferably a switch or dial, for setting the time period.

In one embodiment, the interface 104 is designed for signal communication.

The interface 104 is configured, for example, to receive a signal for deactivating at least the electronic system 102, wherein the controller 103 is configured to deactivate at least the electronic system 102 upon receipt of the signal for deactivating.

The interface 104 is configured, for example, to receive an activation signal for at least part of the electronic system 102, wherein the controller 103 is configured to activate at least part of the electronic system 102 when the activation signal is received.

In one embodiment, the controller 103 is designed to activate or deactivate that part of the electronic system 102 that is activated or deactivated depending on location information.

For example, it is planned to send the deactivation signal or the activation signal depending on location information. In the example, the location information is a statement about a room in which the device 100 is located. For example, the location information is used to avoid unwanted deactivation.

In one embodiment, the controller 103 is designed to determine the location of the device 100 to avoid undesired deactivation. For example, by evaluating the location, an unwanted deactivation due to a temperature above the temperature threshold is avoided, which would otherwise be caused by the effects of sunlight during transport or light sources in an operating room.

In one embodiment, the controller 103 is designed to deactivate at least part of the electronic system 102 by mechanically or electrically interrupting an electrical connection between the power source 101 and at least part of the electronic system 102.

Separating at least the electronic system 102 from the energy source 101 offers various advantages, particularly for use in the field of sterile technology. The fact that the electronic system 102 is without power or partially without power prevents short circuits within the electronic system 102. This is particularly advantageous for the moisture-intensive machine reprocessing steps in sterile supply.

In one embodiment, the controller 103 is designed to deactivate at least the part of the electronic system 102 by switching off a component of the part of the electronic system 102.

In one embodiment, the controller 103 is designed to activate at least part of the electronic system 102 by establishing an electrical connection between the energy source 101 and at least part of the electronic system 102.

The device 100 or the electronic system 102 comprises, for example, a switch for making or breaking the electrical connection.

In one embodiment, the controller 103 comprises a shape memory element. The shape memory element is, for example, a bimetal. The shape memory element is configured, for example, to deactivate at least the part of the electronic system 102 in a first form of the shape memory element by interrupting the electrical connection between the energy source 101 and at least the part of the electronic system 102.

The shape memory element is designed, for example, to activate at least the part of the electronic system 102 in a second form of the shape memory element by establishing the electrical connection between the energy source 101 and at least the part of the electronic system 102.

The shape-memory element is designed, for example, as a switch for making or breaking the electrical connection or is designed to switch the switch for making or breaking the electrical connection.

In one embodiment, the interface 104 comprises a coil. The controller 103 is designed to recognize a voltage induced in the coil and to activate at least the part of the electronic system 102 when the induced voltage is recognized. The coil is, for example, a coil for near-field communication, preferably an NFC tag or RFID tag.

In one embodiment, the controller 103 is designed to switch the switch to make or break the electrical connection depending on the induced voltage.

In one embodiment, the controller 103 comprises a switching element. The circuit element is designed to activate at least part of the electronic system 102.

The switching element is arranged on the device 100 in such a way that the switching element is in communication or contact with a sterile goods container for the sterile goods during the process.

The switching element is designed to deactivate at least the part of the electronic system 102 when contact with the sterile container is lost.

In one embodiment, the switching element, in a first position of the switching element, allows a current provided by the energy source 101 to flow through the switching element and, in a second position of the switching element, prevents a current from flowing through the switching element.

In one embodiment, the controller 103 is designed to distinguish the first position of the switching element from the second position of the switching element and to activate at least part of the electronic system 102 when the first position or a change from the second position to the first position is detected.

In one embodiment, the switching element is designed to be in the second position when it comes into contact with the sterile supply container and to move to the first position when contact with the sterile supply container is lost.

FIG. 2 schematically shows a plot 200 of pressure p over time t in a process for washing or sterilizing sterile goods, using the example of steam sterilization.

In the example, the pressure p moves in different phases below 1 bar and above 1 bar to 3.1 bar:
201 Fractional vacuum
202 Ascent time
203 Sterilization time
204 Pressure drop
205 Fractional drying
206 Ventilation

The phases of fractional vacuum 201 and rise time 202, for example, last until the contaminated air is removed or the temperature for the sterilization time 203 is reached. The sterilization time of 203 is at least 3 minutes at a temperature of 134° C. The fractional drying 205 and ventilation 206 are part of drying time.

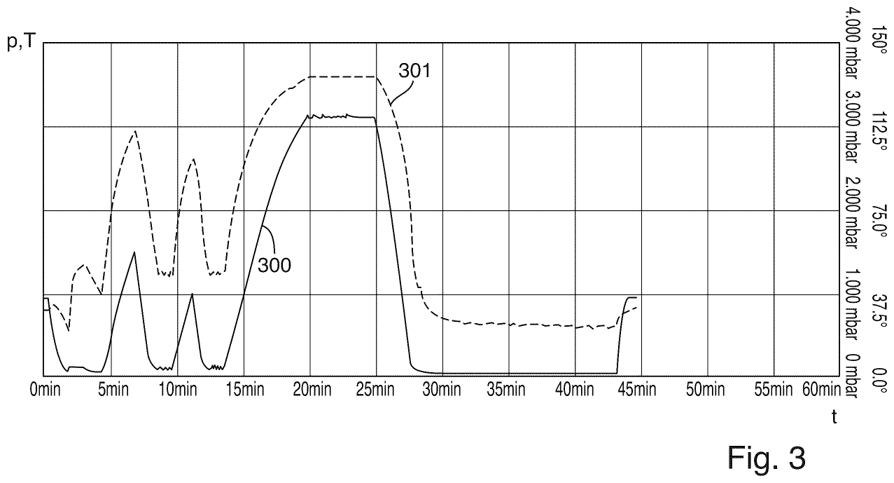

FIG. 3 shows a graph 300 of pressure p between 0 millibar and ˜3150 millibar (0 to 3.15*10\hat{ }5 Pa) and a graph of temperature T between room temperature and 135° Celsius for 45 minutes of a process for sterilizing sterile goods, using the example of steam sterilization with a fractionated vacuum and a drying time of 15 minutes at the end of the process.

High temperatures and in particular the maximum temperature of 135° Celsius reached and maintained in the process are critical for the energy source 101. In the example, to protect the energy source 101, provision is made for disabling at least part of the electronic system 102, at least in the area of the operation in which the temperature is 135° Celsius or essentially 135° Celsius. This means that the temperature threshold for the exemplary curve is set lower than 135° Celsius, preferably between 70° Celsius and 80° Celsius.

If moisture developing during the process is critical for the electronic system 102, it can be reduced during the drying time.

In one example, the time that passes before the electronic system 102 is activated is chosen so that the activation occurs at the end of the drying time. This means that the sterilization time 203, the time of the pressure drop 204 and the drying time 30 minutes after the start of the process are waited for in the exemplary process.

Activation can be particularly advantageous after a cooling period, for example 30 minutes after the end of the drying time.

Different sterilization processes with different temperatures or pressures or durations of the phases or a different composition of the phases may be provided for different sterile goods or autoclaves or hospitals. The temperature threshold and the time waited before activation are adjusted accordingly.

Different temperature thresholds can be set for activation and deactivation. For example, the temperature threshold for activation is lower than for deactivation.

In an example in which the controller 103 comprises the shape memory element, at least part of the electronic system 102 is activated by establishing the electrical connection between the energy source 101 and at least part of the electronic system 102. In an example in which the controller 103 comprises the shape memory element, at least the part of the electronic system 102 is deactivated by interrupting the electrical connection between the energy source 101 and at least the part of the electronic system 102.

In an example in which the controller 103 comprises the switching element, at least part of the electronic system 102 is activated upon contact between the switching element and the sterile goods container. For example, the switching element moves to the first position when contact is made, thereby establishing the electrical connection between the energy source 101 and at least part of the electronic system 102.

In an example in which the controller 103 comprises the switching element, at least the part of the electronic system 102 is deactivated when contact between the switching element and the sterile goods container is lost. For example, if contact is lost, the switch moves to the second position, thereby interrupting or disconnecting the electrical connection between the power source 101 and at least part of the electronic system 102.

The controller 103 is designed to execute a method for operating the device 100 in an example.

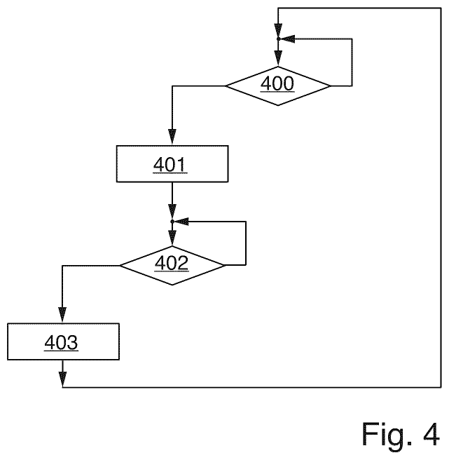

FIG. 4 is a flowchart illustrating steps in the method of operating the device 100.

In the example at the beginning of the procedure, the device 100 is in a state in which the entire electronic system 102 is activated.

The procedure includes a step 400.

In step 400, it is checked whether or not a process for washing or sterilizing is recognized.

If the process is recognized, a step 401 is executed. Otherwise, step 400 is executed. Preferably, this is done by repeating step 400 with a certain delay.

The process is recognized depending on the temperature detected.

For example, the temperature is recorded and it is determined that at least the part of the electronic system 102 is to be deactivated if the temperature reaches or exceeds the temperature threshold.

It may be envisaged that the signal for deactivating at least part of the electronic system 102 is received in step 400, and that upon receipt of the signal for deactivating, it is determined that at least part of the electronic system 102 is to be deactivated.

In an example in which the controller 103 comprises the switching element, at least part of the electronic system 102 is activated upon contact between the switching element and the sterile goods container. For example, the switching element moves to the first position when the contact is made, causing the contact to be recognized by the controller 103 and the controller 103 to activate at least the part of the electronic system 102.

In step 401, at least the part of the electronic system 102 is deactivated in the process.

The part of the electronic system 102, for example, is activated when the sterilization process is detected or after the time has elapsed after the sterilization process is detected.

The deactivating signal may be arranged to include location information, wherein the part of the electronic system 102 that is deactivated is deactivated in dependence on the location information.

In one example, at least the part of the electronic system 102 is deactivated by switching off a component of the electronic system 102.

In one example, at least part of the electronic system 102 is deactivated by mechanically or electrically interrupting the electrical connection between the energy source 101 and at least part of the electronic system 102.

After that, a step 402 is executed.

Step 402 checks whether at least part of the electronic system 102 is to be activated or not.

If at least the part of the electronic system 102 is to be activated, a step 403 is executed. Otherwise, step 402 is executed.

In one example, other than disabling part of the electronic system 102 to sense temperature, wherein if the temperature is determined to be below the temperature threshold, the disabled part of the electronic system 102 is determined to be enabled.

It may be provided that the signal for activating at least part of the electronic system 102 is received in step 402 and that, upon receiving the signal for activation, it is determined that at least part of the electronic system 102 is to be activated.

The drying processes of cleaning and disinfection and the sterilization process are used to eliminate potential residual moisture in the electronic system 102 prior to activation by appropriately designing the temperature threshold for the temperature, the time period and/or a point in time at which the signal is sent to activate.

In step 403, at least the part of the electronic system 102 is activated.

In one example, at least the part of the electronic system 102 is activated by turning on the component of the electronic system 102.

In one example, at least part of the electronic system 102 is activated by mechanically or electrically establishing the electrical connection between the energy source 101 and at least part of the electronic system 102.

In an example in which interface 104 includes the coil, the voltage induced in the coil is detected, and at least part of the electronic system 102 is activated upon detection of the induced voltage and/or by the induced voltage.

In an example in which the controller 103 comprises the switching element, at least the part of the electronic system 102 is activated when contact between the switching element and the sterile goods container is lost. For example, if the switching element moves to the first position, the loss of contact is detected by the controller 103 and at least the part of the electronic system 102 is activated by the controller 103.

It may be provided that the signal for activation is dependent on the location information.

After that, step 400 is executed in the example. It may be stipulated that the procedure ends after step 403.

The method can provide that the temperature threshold for the temperature and/or the time until its expiration before deactivating at least the part of the electronic system is waited for and/or the time period over the interface 104, i.e. for example over the data communication interface or the mechanical element, i.e. the switch or control dial, is predefined.

1. A device for locating sterile goods, the device comprising:
an electronic system for transmitting a locating signal;
a power source configured to supply the electronic system with energy; and
a controller configured to recognize a washing or sterilizing process and to at least temporarily deactivate at least a part of the electronic system in the washing or sterilizing process.

2. The device according to claim 1, wherein the controller is configured to detect a temperature and to deactivate the part of the electronic system when the temperature reaches or exceeds one of the following:
a predetermined temperature threshold value, or
a temperature threshold value that is presettable.

3. The device according to claim 2, wherein the controller is configured to activate the part of the electronic system when the temperature reaches or is less than one of the following:
the predetermined temperature threshold value, or
the temperature threshold value that is presettable.

4. The device according to claim 1, further comprising an interface for signal communication, the interface configured to receive an activation signal for activating the part of the electronic system and/or receive a deactivation signal for deactivating the part of the electronic system, wherein the controller is configured to deactivate the part of the electronic system upon receiving the deactivation signal and/or activate the part of the electronic system upon receiving the activation signal.

5. The device according to claim 1, wherein the controller is configured to activate the part of the electronic system after a predetermined time or a time that is predeterminable via an interface of the device for setting the time after detection of an event.

6. The device according to claim 1, wherein the controller comprises a shape memory element configured to assume at least a first shape and a second shape depending on temperature, wherein the shape memory element is configured to deactivate the part of the electronic system in a first shape of the shape memory element by interrupting an electrical connection between the power source and the part of the electronic system, and to activate the part of the electronic system by establishing the electrical connection between the power source and the part of the electronic system in a second form of the shape memory element.

7. The device according to claim 6, wherein the shape memory element comprises a bi-metal.

8. The device according to claim 1, further comprising an interface for setting a time after detection of an event, wherein the interface comprises a coil, and wherein the controller is configured to detect a voltage induced in the coil and activate the part of the electronic system upon detection of the voltage.

9. The device according to claim 8, wherein the coil is configured for near-field communication.

10. The device according to claim 9, wherein the coil is an NFC tag or RFID tag.

11. The device according to claim 1, wherein the controller comprises a switching element configured to activate the part of the electronic system, wherein the switching element is arranged on the device in such a way that the switching element is in communication or contact with a sterile goods container for the sterile goods during the washing or sterilizing process, wherein the switching element is configured to activate the part of the electronic system in response to a loss of contact with the sterile goods container.

12. The device according to claim 1, wherein the electronic system comprises the part of the electronic system and a second part, wherein the part of the electronic system is open and/or configured to be flushed through, and wherein the second part is encapsulated.

13. The device according to claim 1, wherein the electronic system is open and/or configured to be flushed through and the power source is encapsulated.

14. A method for operating a device for locating sterile goods, the device comprising a power source and an electronic system, the method comprising the steps of:
supplying the electronic system with energy from the power source;
detecting a washing or sterilizing process; and
deactivating at least a part of the electronic system at least temporarily in the washing or sterilizing process.

15. The method according to claim 14, further comprising the step of detecting a temperature and deactivating the part of the electronic system when a temperature threshold value is reached or exceeded.

16. The method according to claim 15, wherein:
the part of the electronic system is activated when the temperature falls below the temperature threshold value, and
the part of the electronic system is deactivated when the temperature reaches or exceeds the temperature threshold value.

17. The method according to claim 15, wherein:
the part of the electronic system is activated when the temperature falls below the temperature threshold value, and
the part of the electronic system remains activated when the temperature reaches or exceeds the temperature threshold value.

18. The method according to claim 14, further comprising at least one of the following steps:
receiving an activation signal for the part of the electronic system and activating the part of the electronic system upon receipt of the activation signal; and
receiving a deactivation signal for the part of the electronic system and deactivating the part of the electronic system upon receipt of the deactivation signal.

19. The method according to claim 14, wherein the part of the electronic system is activated for sterilization only after a time after detection of an event.

20. The method according to claim 14, wherein:
the device comprises a coil,
a voltage induced in the coil is detected; and
the part of the electronic system is activated upon detection of the voltage.
